(19) United States
(12) Patent Application Publication
(10) Pub. No.: US 2011/0190858 A1
(43) Pub. Date: Aug. 4, 2011

(54) LEAD HAVING EXPANDABLE DISTAL PORTION

(75) Inventors: Martin T. GERBER, Maple Grove, MN (US); Michael D. Baudino, Coon Rapids, MN (US)

(73) Assignee: Medtronic, Inc., Minneapolis, MN (US)

(21) Appl. No.: 13/011,418

(22) Filed: Jan. 21, 2011

Related U.S. Application Data
(60) Provisional application No. 61/299,569, filed on Jan. 29, 2010.

(51) Int. Cl. A61N 1/05

(52) U.S. Cl. 607/116

(57) ABSTRACT

Leads may include expandable and collapsible distal end portions that provide anchoring in tissue when expanded and allow for insertion through an introducer when collapsed.

### RELATED APPLICATION

This patent application claims priority to U.S. Provisional Patent Application Ser. No. 61/299,674, filed Jan. 29, 1010, the entire disclosure of which is expressly incorporated herein by reference.

### FIELD

The present disclosure relates generally to implantable medical leads, particularly leads having expandable distal portions for anchoring the lead in tissue when implanted in a patient, and related systems and methods.

### BACKGROUND

A variety of implantable medical devices have been proven to be effective for treatment of a variety of diseases. Many of such devices, such as cardiac pacemakers, defibrillators, spinal cord or deep brain stimulators, gastric stimulators, and the like, employ accessory medical leads to deliver electrical signals from a signal-generating device to tissue of a patient at a location removed from the signal generating device. Typically the lead is tunneled from a subcutaneous region of the patient in which the signal generating device is implanted to a target tissue location.

It is often important that the lead, or portions thereof, does not shift or move once implanted to ensure that a therapeutic signal continues to be delivered to the target tissue. One mechanism for retaining the implanted position of a lead or portion thereof is the use of tines. The tines are typically attached to various locations of the lead and are deployed once the lead is properly positioned in the patient. Most often, tines prevent retrograde movement of the lead. However, once the tines are deployed, it can be difficult to change the position of the lead.

Prior to deploying the tines, it is often desirable to apply electrical signals to the patient via electrodes of the lead, as the lead is being implanted, to determine whether the lead is being positioned in an appropriate location or if the tract of implantation is proceeding in a desired direction. This process is sometimes referred to a trolling, where test electrical signals are applied as the lead is advanced to aid in the proper placement of the lead. However, with the use of standard lead introducer devices, it is not possible to perform such trolling when the tines are disposed on the lead distal to the electrodes. That is, absent tines being distal electrodes of the lead, the lead may extended distally beyond the introducer (or the introducer may be withdrawn to expose the distal end of the lead) such that a test electrical signal may be delivered to the patient via electrodes of the lead, and the lead may be withdrawn into the introducer (or introducer advanced) and repositioned. This process may be repeated until the lead is determined to be in an appropriate location, and the introducer may be completely withdrawn. However, when the tines are disposed on the lead distal to the electrodes, the tines will be deployed during the initial test stimulation (when extended beyond the distal end of the introducer), and the ability to reposition the lead will be compromised, if not lost.

### SUMMARY

This disclosure, among other things, describes implantable medical leads having deployable anchoring mechanisms that may be retracted so that the lead may be withdrawn back into an introducer and repositioning may occur.

In various embodiments, a lead includes a proximal body having a longitudinal axis and a distal portion extending from the proximal portion. The distal portion has a changeable width and includes a central body portion including a plurality of electrodes aligned with the longitudinal axis of the proximal body. The distal portion also includes first and second edge portions on opposing sides of the central body portion. The edge portions define the width of the distal portion and have a collapsed configuration and an expanded configuration. When the first and second edge portions are in the collapsed configuration, the distal portion of the lead has a first width and is configured to be received by a lumen of an introducer. When the first and second edge portions are biased towards the expanded configuration, and when in the expanded configuration the distal portion of the lead has a second width greater than the first width, and the second width is greater than or equal to an outer diametric dimension of the introducer. The first and second edge portions are free of electrodes. The first and second edge portions, in the expanded configuration, may have (i) ramped a distal portions configured to facilitate conversion to the collapsed configuration as the distal portion is pushed into the lumen of the introducer, and (ii) ramped proximal portions configured to facilitate conversion to the collapsed configuration as the distal portion is pulled into the lumen of the introducer, allowing retraction of the lead into the introducer for repositioning of the lead during an implant procedure.

In various embodiments, an implantable medical lead includes an elongate body member having a proximal end and a distal end, a plurality of contacts in proximity to the proximal end, and a plurality of electrodes in proximity to the distal end. Each of the plurality of electrodes is electrical coupled to a discrete contact of the plurality of contacts. The lead also includes a plurality of protrusions extending from the body in proximity to the distal end. The protrusions are resilient and have a collapsed configuration and an expanded configuration. The protrusions are biased in the expanded configuration. The elongate body member has a diametric dimension smaller than the inner diametric dimension defined by a lumen of an introducer such that the body member is capable of being slidably received by the lumen of the introducer. The lead, in the distal region having the plurality of protrusions, has (i) a diametric dimension greater than or equal to the outer diametric dimension of the introducer when the protrusions are in the expanded configuration, and (ii) a diametric dimension less than the inner diametric dimension of the introducer when the protrusions are in the collapsed configuration. The protrusions have ramped distal portions configured to facilitate conversion to the collapsed configuration as the distal portion is pushed into the lumen of the introducer, and have ramped proximal portions configured to facilitate conversion to the collapsed configuration as the distal portion is pulled into the lumen of the introducer, allowing retraction of the lead into the introducer for repositioning of the lead during an implant procedure.

One or more embodiments described herein provide one or more advantages over prior leads, devices, systems and methods where the leads employ distal anchoring mechanisms such as tines. Such advantages will be apparent to those of skilled in the art upon reading the following detailed description.

### BRIEF DESCRIPTION OF THE DRAWINGS

The accompanying drawings, which are incorporated into and form a part of the specification, illustrate several embodiments of the present disclosure and, together with the description, serve to explain the principles of the disclosure. The drawings are only for the purpose of illustrating embodiments of the disclosure and are not to be construed as limiting the disclosure.

FIGS. 1-3 are schematic plan views of embodiments of leads having expandable distal portions.

FIGS. 4A-B are schematic sectional views of embodiments of edge portions of a lead.

FIG. 5A-D are schematic sectional views of an introducer showing a lead before insertion into (5A), inserted in (5B), after passage through (5C), and after retraction into (5D) the introducer.

FIG. 6 is a schematic plan view of an embodiment of a lead having an expandable distal portion.

FIGS. 7A-B are schematic close-up views of alternative embodiments of a portion of the lead in FIG. 6 showing a protrusion.

FIG. 8 is a schematic plan view of an embodiment of a lead having protrusions at the distal end portion.

FIGS. 9A-B are schematic sectional views of an introducer showing a lead disposed within (9A) and after passage through (9B) the introducer.

FIG. 10 is a schematic view of a representative implantable electrical signal therapy system.

FIG. 11 is a schematic representation of an exemplary spinal cord stimulation (SCS) system implanted in a patient.

FIG. 12 is a schematic representation of an exemplary bifurcated lead implanted in a patient.

The schematic drawings presented herein are not necessarily to scale. Like numbers used in the figures refer to like components, steps and the like. However, it will be understood that the use of a number to refer to a component in a given figure is not intended to limit the component in another figure labeled with the same number. In addition, the use of different numbers to refer to components is not intended to indicate that the different numbered components cannot be the same or similar.

### DETAILED DESCRIPTION

In the following detailed description, reference is made to the accompanying drawings that form a part hereof, and in which are shown by way of illustration several embodiments of devices, systems and methods. It is to be understood that other embodiments are contemplated and may be made without departing from the scope or spirit of the present disclosure. The following detailed description, therefore, is not to be taken in a limiting sense.

All scientific and technical terms used herein have meanings commonly used in the art unless otherwise specified. The definitions provided herein are to facilitate understanding of certain terms used frequently herein and are not meant to limit the scope of the present disclosure.

As used in this specification and the appended claims, the singular forms “a”, “an”, and “the” encompass embodiments having plural referents, unless the content clearly dictates otherwise.

As used in this specification and the appended claims, the term “or” is generally employed in its sense including “and/or” unless the content clearly dictates otherwise. As used herein, “have”, “having”, “include”, “including”, “comprise”, “comprising” or the like are used in their open ended sense, and generally mean “including, but not limited to.”

“Exemplary” or “representative” is used in the sense of “for example” or “for the purpose of illustration”, and not in a limiting sense.

As used herein, “aligned”, as it relates to aligning one or more electrodes an axis, means that at least a portion of each of the one or more electrodes overlaps with a plane through the axis.

In various embodiments, this disclosure, among other things, describes implantable medical leads having deployable anchoring mechanisms that may be retracted so that the lead may be withdrawn back into an introducer and repositioning may occur. The deployable anchoring mechanisms are located in proximity to electrodes at the distal portion of the lead and can serve to prevent undesired movement or migration of the electrodes relative to the tissue in which they are implanted.

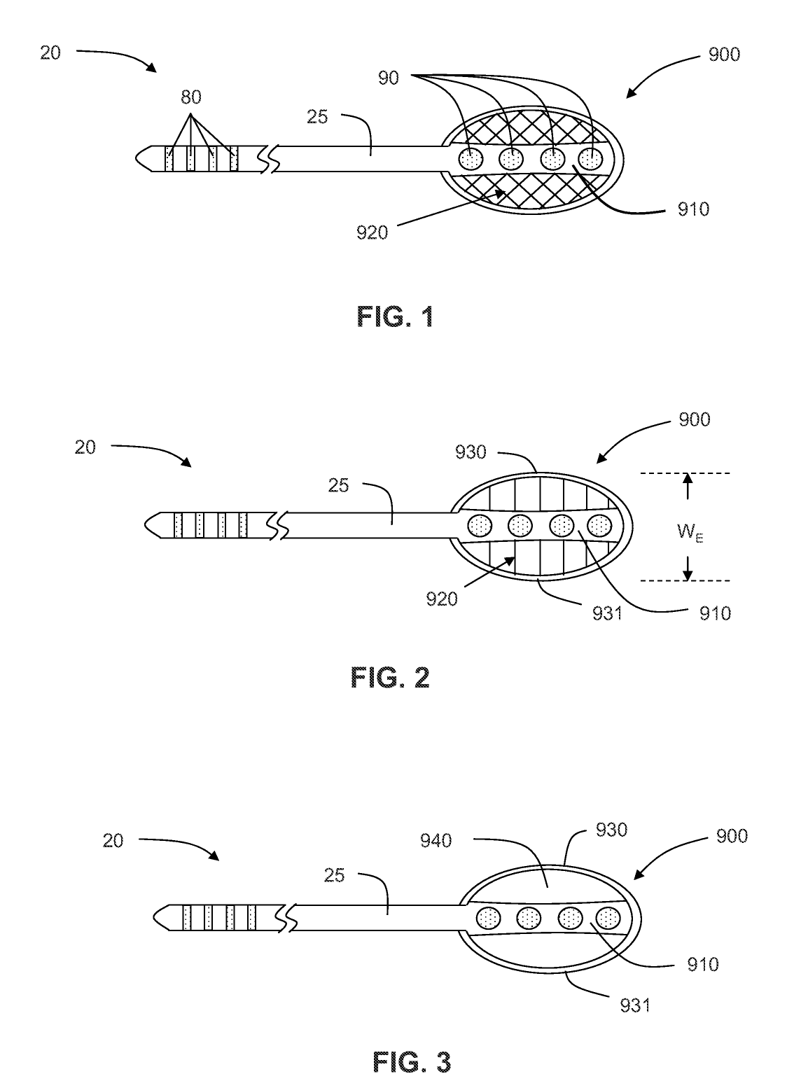

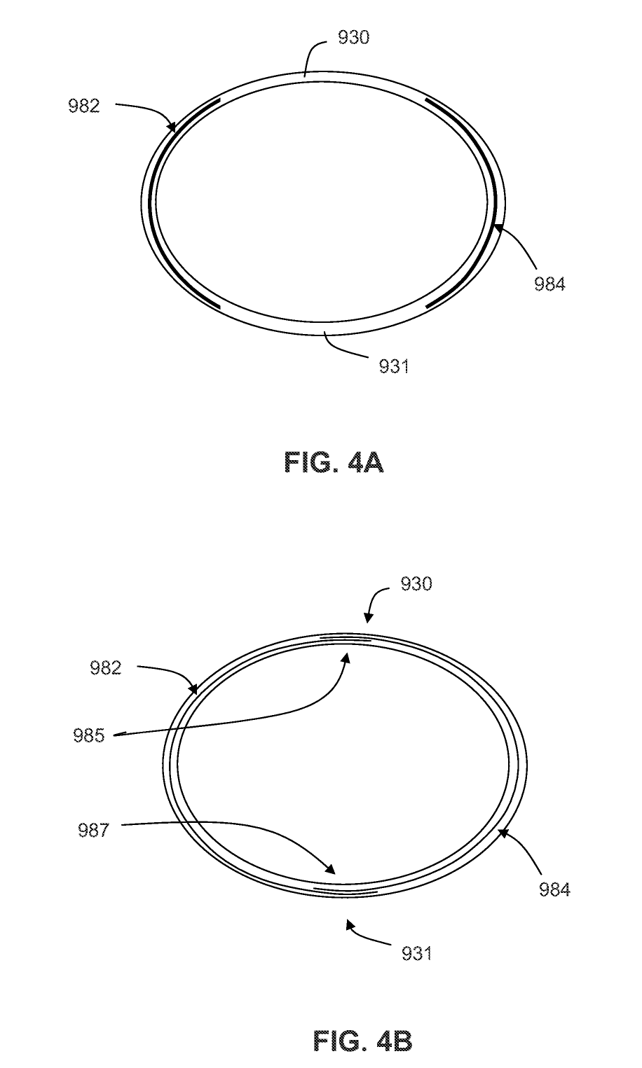

Referring now to FIGS. 1-3, embodiments of leads 20 having distal anchoring mechanisms are shown. The leads 20 include a proximal body 25 having an axis, and a distal portion 900 extending from the proximal body portion 25. The distal portion has a changeable width, as described in more detail below with regard to, e.g., FIG. 4. The distal portion 900 of the lead 20 includes a central body portion 910 that includes a plurality of electrodes 90. Each of the electrodes 90 is electrically coupled with a discrete contact 80 in proximity to the proximal end of the lead. The electrodes 90 are aligned with the longitudinal axis of the proximal body member 25. The depicted electrodes 90 may be similar to those employed with paddle-type leads, where the electrodes are exposed on one face of the paddle. While not shown, it will be understood that ring-type electrodes that would extend around the central body portion or other electrode types may be employed.

The distal portion 900 of the lead 20 also includes first 930 and second 931 edge portions that define the width of the distal portion 900. The edge portions 930, 931 have expanded and collapsed configurations, e.g. as will be discussed in more detail below with regard to, e.g., FIG. 4. In FIGS. 1-3, the edge portions 930, 931 are in their expanded configurations and the distal portion 900 has an expanded width (W_{E}). The first 930 and second 931 edge portions are on generally opposing sides of the central body portion 910. In some embodiments, the first 930 and second 931 edge portions are contiguous. In some embodiments, the first 930 and second 931 edge portions are integrally formed with the central body portion 910. The proximal body 25 and central body may be constructed in a manner similar to construction of paddle-type leads or percutaneous leads.

The distal portion 900 of the lead 20 also includes one or more resilient members 920 that span the gap between the central body 910 and the first edge portion 930 and the central body 910 and the second edge portion 931. The resilient members 920 bias the first 930 and second 931 edge portions towards the expanded configurations. The resilient members 920 may be formed from a shape-memory metal or alloy, such as nitinol, or any suitable biocompatible polymer, such as nylon, polyurethane, polycarbonate, or the like. In some embodiments, the resilient member(s) 920 are compressible foam. The resilient members 920 can be bent or folded, as desired or needed, but in the absence of external folding or bending forces, or the like, assume a position that causes the first 930 and second 931 edge portions to assume the expanded configuration. The resilient members 920 may be integrally formed with the central body 910 or the first 930 and second 931 edge portions or may be bonded, adhered, welded, affixed or otherwise operably coupled to the central body 910 and the first 930 and second 931 edge portions. The resilient members 920 may take any suitable form, such as a web (see FIG. 1), columns or bars (see FIG. 2), or the like.

With reference to FIG. 3, a flexible membrane 940 spans the area between the central body portion 910 and the first edge portion 930, and a second flexible membrane spans the area between the central body portion 910 and the second edge portion 931. Such a membrane 940, which may be formed from any suitable material such as silicone, may prevent tissue in growth so that the lead 20 may be explanted or repositioned after implant. Of course, in some circumstances, it may be desirable to omit such a membrane as it may be desirable to use tissue in growth as a mechanism for further stabilizing the position of the electrodes 90 in the tissue in which they are implanted. Despite the lack of a membrane, short term repositioning of the distal portion of the lead; e.g. for the purposes of trolling, may still be accomplished.

The first and second edge portions 930, 931 may be formed of any suitable material such as a biocompatible polymer material. The edge portions 930, 931 may be formed separately or may be formed as a single unit or piece. In some embodiments, the edge portions and the central portion 910 are formed from a single unit or piece. The edge portions 930, 931 may be of solid or hollow core constructions and may be molded, extracted or the like. In embodiments, where the edge portions 930, 931 are formed from one or more units separate from the central body portion 910 or the body member 25, the edge portions may be attached to the central portion 910 or the body portion 25 via adhesive, bonding, welding, or the like.

Referring now to FIG. 4A, embodiments of edge portions 930, 931 having incorporated biasing elements 982, 984 are shown in schematic sectional views. In the depicted embodiments, the edge portions 930, 931 are contiguous and are formed of a hollow tube into which biasing elements 982, 984 are placed. The depicted biasing elements 982, 984 are biased to a straight configuration, thus they bias the edges 930, 931 to the expanded configuration as depicted. In the embodiments depicted in FIG. 49, the biasing elements 982, 984 overlap one another within a region of the first and second edges (the areas of overlap are depicted at regions 985 and 987). The biasing elements may be formed of shape-memory or resilient materials as described above. The biasing elements 982, 984 may be in any suitable form, such as a bar, rod, strip. In some embodiments, the biasing elements 982, 984 are in configured in a manner similar to a biasing portion of a safety pin (not shown). The distal portion of the contiguous edge assemblies shown in FIGS. 4A-B may be attached to the distal portion of the central member 910 (see, e.g., FIGS. 1-3) via any suitable mechanism, such as adhesive, welding, bonding, or otherwise affixing. The proximal portion of the contiguous edge assemblies may be attached to the proximal portion of the central member 910 or the body member 25.

In the leads 20 depicted in FIGS. 1-3, the electrodes 90 are restricted to placement aligned with the axis of the proximal body 25 and the first 930 and second 931 edge portions are free from electrodes.

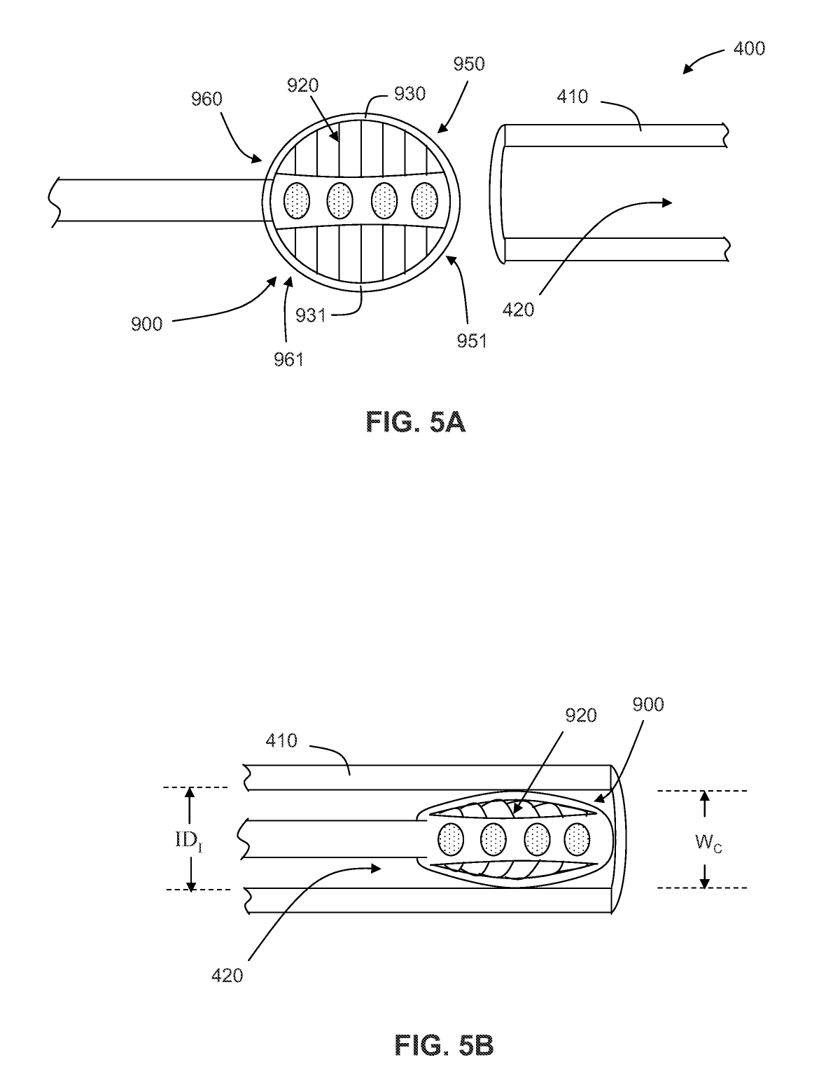

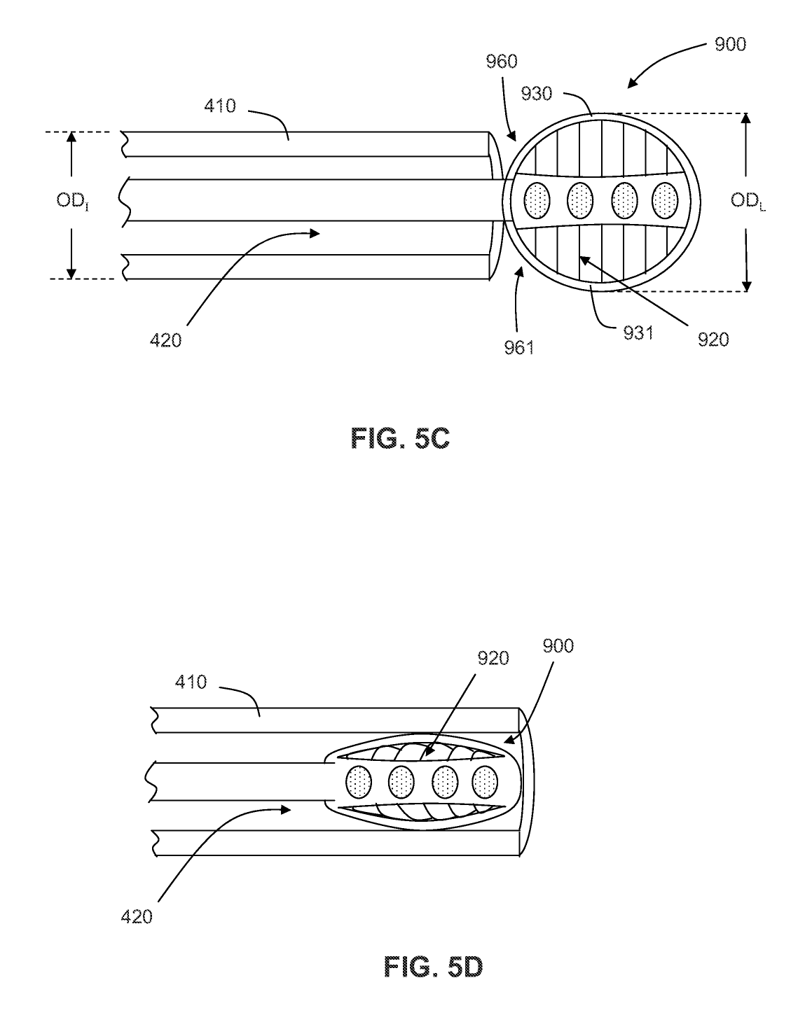

Referring now to FIGS. 5A-D, views a distal portion 900 of a before insertion into (5A), inserted in (5B), after passage through (5C), and after retraction into (5C) an introducer 400 are shown. The introducer 400 has a body member 410 defining a lumen 420. The body member 410 has an inner diametric dimension (ID_{I}) defined by the lumen 420 and an outer diametric dimension (OD_{I}). In some embodiments, the introducer is steerable. Examples of steerable introducers that may be used or modified in accordance with the teaching presented herein include those described in U.S. Pat. No. 7,037,290 to Gardeski, entitled “Multi-Lumen Steerable Catheter,” issued May 2, 2006; U.S. Pat. No. 6,059,739 to Baumann, entitled “Method and Apparatus for Deflecting a Lead or Catheter,” issued May 2000; U.S. Pat. No. 6,836,687 to Kelley, entitled “Method and System for Delivery of a Medical Electrical Lead Within a Venous System,” issued Dec. 28, 2004; or the like.

In FIGS. 5B and 5D, the distal portion 900 of the lead (and thus the edge portions) is in a collapsed configuration and has a collapsed width (W_{C}) and is configured to be received by and slidably disposed in the lumen 420 of the introducer. In FIGS. 5A and 5C, the distal portion 900 of the lead (and thus the edge portions) is in the expanded configuration and has a width (W_{E}) greater than the collapsed width and greater than the outer diameter of the introducer (OD_{I}). As the introducer 400 is advanced through a patient, a track having an inner diameter similar to the outer diameter of the introducer is formed. By having a width greater than the outer diameter of the introducer body 410, the distal portion 900 of the lead may anchor into tissue in which it is implanted, via pressing of the edge portions of the distal portion of the lead against the walls of the track in the tissue due to the resilient nature of the resilient members.

As shown in FIG. 5A, the first 930 and second 931 edge portions may include distal ramped or curved regions 950, 951 that facilitate conversion of the distal portion 900 of the lead from the expanded configuration to the collapsed configuration as the distal portion 900 is pushed into the lumen 420 of the introducer. As shown in FIG. 5C, the first 930 and second 931 edge portions may include proximal ramped or curved regions 961), 961 that facilitate conversion of the distal portion 900 of the lead from the expanded configuration to the collapsed configuration as the distal portion 901) is pulled into the lumen 420 of the introducer.

As the ramped portions 950, 951, 960, 961 are pushed or pulled against the proximal or distal end of the body member 410 of the introducer, the force of pushing or pulling, along with the ramped configuration of the edge portions 931), 931 causes the edges to assume a collapsed configuration, allowing the distal portion 900 of the lead to enter the lumen 420 of the introducer. Preferably, the lead body is formed of material and constructed in a way such that the lead is sufficiently pushable to allow the distal portion 900 of the lead to collapse and enter the lumen when the lead body is pushed against the body of the introducer. Well-known lead manufacturing techniques and materials may be employed to impart such pushability. Of course, the lead body may have a lumen open to the proximal end to allow a stylet to enter and push the lead if the lead body does not have sufficient pushability.

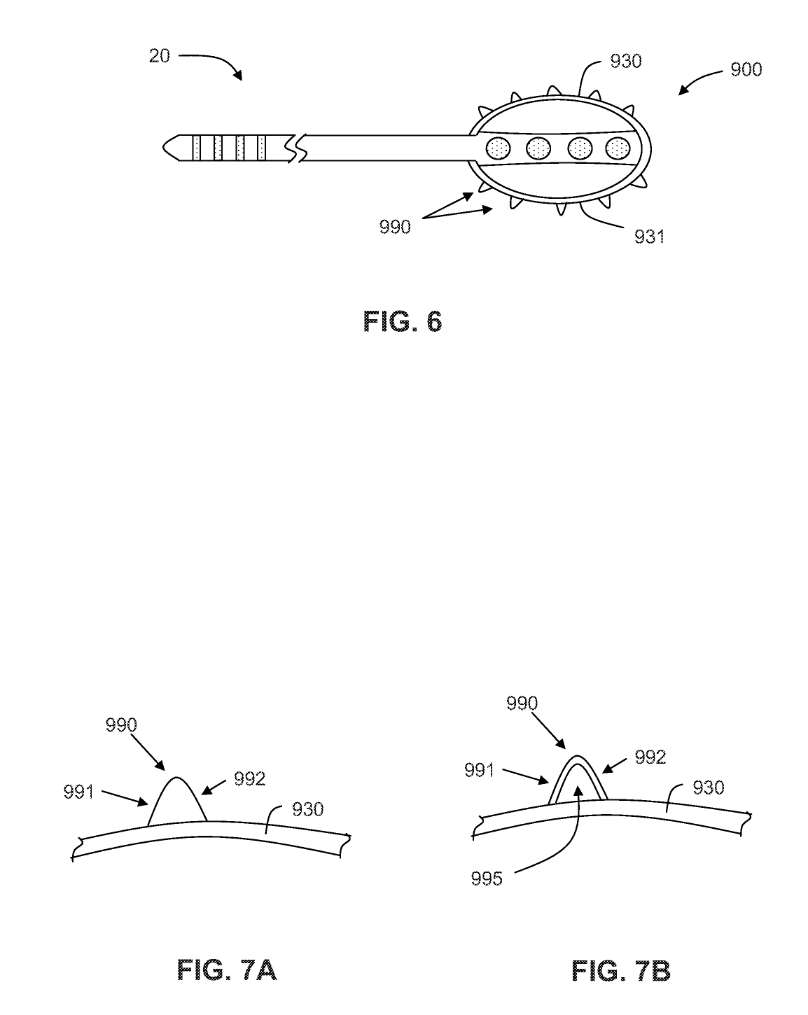

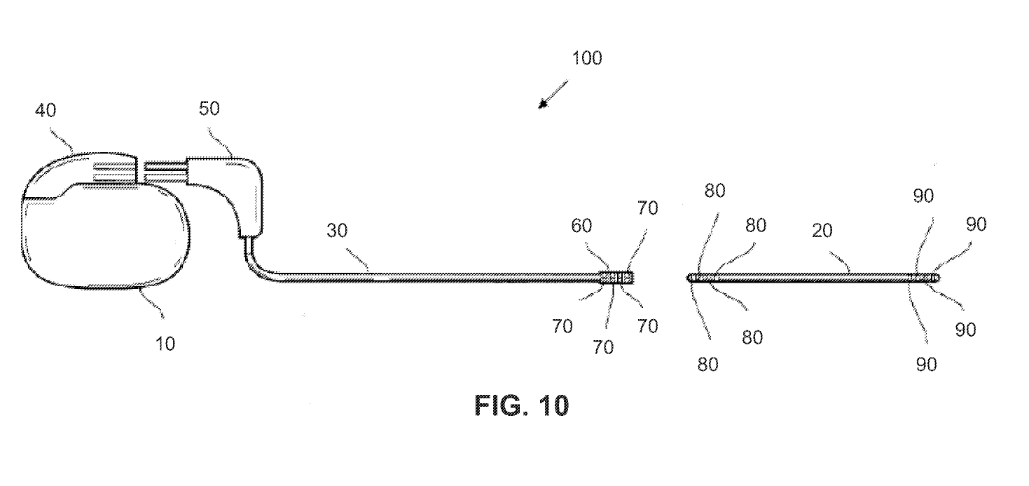

Referring now to FIG. 6, a lead 20 similar to the lead shown in FIG. 3 is depicted. The distal portion 900 of the lead 20 in FIG. 10 has a plurality of protrusion disposed along the first 930 and second 931 edge portions. The protrusions 990 may enhance the anchoring ability of the distal portion 900 once deployed in tissue of a patient. The protrusions 990 may integrally formed with or welded, bonded, adhered or otherwise affixed to the edge portions 930, 931. The protrusions 990 may be formed of any suitable material, such as a metallic or polymeric material, including any suitable biocompatible polymeric material in some embodiments, the protrusions are formed of silicone.

Referring now to FIGS. 7A-B, close-up views of embodiments of protrusions 990 (e.g., as depicted in FIG. 6) are shown. The protrusion 990 have a proximal ramped portion 991 and a distal ramped portion 992 to facilitate entry into the lumen of an introducer (e.g., as described above with regard to the ramped portions of the edge portions).

In some embodiments, the protrusions 990 are collapsible and expandable. For example the protrusions 990 may be of solid core construction (FIG. 7A) and be formed of, for example, compressible foam. In some embodiments, the protrusions 990 are hollow or otherwise have a cavity 995 (FIG. 7B) that allows for deformation and collapse of the protrusion. The protrusions 990 may be formed of resilient material biased towards the expanded configuration (e.g., the configuration depicted in FIGS. 7A-B, for example). The protrusions may contain biasing elements, as described above with regard to FIGS. 4A-B, for example. Like with the edge portions, the protrusions 990 may, in some embodiments, collapse when pushed or pulled into a lumen of an introducer.

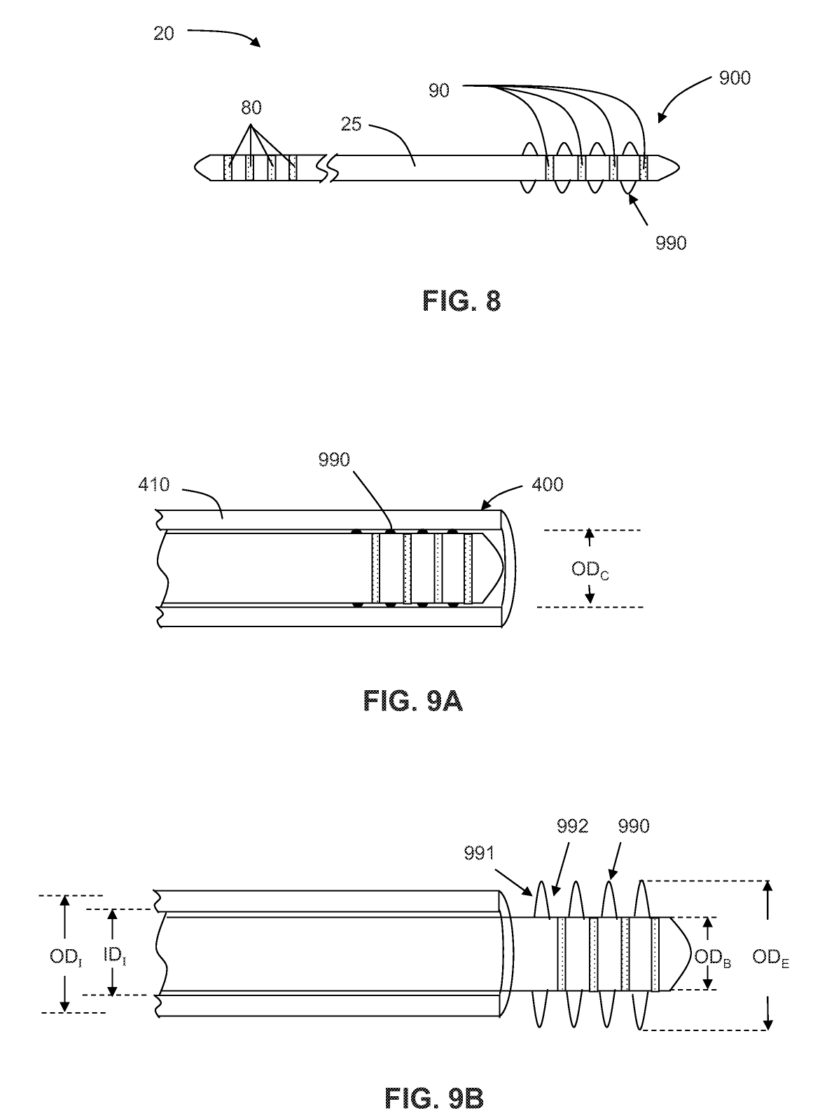

Referring now to FIGS. 8-9, an alternative embodiment of a lead 20 is shown. In FIG. 8 a schematic plan view of the lead 20 is shown. In FIGS. 9A-B schematic sectional views of an introducer 400 and the lead, or a portion thereof, in the lumen of the introducer 400 are shown. The lead 20 has an elongate body member 25 having a proximal end and a distal end. The elongate body 25 has a generally uniform outer diametric dimension and may be generally cylindrical in shape.

A plurality of contacts 80 are in proximity to the proximal end, and a plurality of electrodes 90 are in proximity to the distal end. Each of the plurality of electrodes 90 is electrical coupled to a discrete contact 80. The distal end portion 900 of the lead includes a plurality of protrusions 990 extending from the body 25. The protrusions 990 are resilient and have a collapsed configuration (see FIG. 9A) and an expanded configuration (see FIGS. 8 and 9B). The protrusions 990 are biased in the expanded configuration. The elongate body member 25 has a diametric dimension OD_{B} less than the inner diametric dimension (ID_{I}) defined by a lumen of an introducer body member 410 such that the body member 25 of the lead is capable of being slidably received by the lumen of the introducer 400. The distal region 900 of the lead has (i) a diametric dimension (OD_{E}) greater than the outer diametric dimension (OD_{I}) of the introducer when the protrusions 990 are in the expanded configuration, and (ii) a diametric dimension (OD_{c}) less than or equal to the inner diametric dimension (ID_{I}) of the introducer when the protrusions 990 are in the collapsed configuration. The protrusions 990 have ramped distal portions 992 configured to facilitate conversion to the collapsed configuration as the distal portion 900 is pushed into the lumen of the introducer 400, and have ramped proximal portions 991 configured to facilitate conversion to the collapsed configuration as the distal portion is pulled into the lumen of the introducer 400.

The lead body, contacts, electrodes, and protrusions of the lead shown in FIGS. 8-9 may be formed essentially as described above with regard the leads depicted in FIGS. 1-7.

It will be understood that the leads described herein may be used for any suitable purpose. A general overview of systems that may employ such leads is provided in FIGS. 10-12. For the purpose of convenience, some details regarding the distal portions of the leads are not shown in FIGS. 10-12.

Referring to FIG. 10, a schematic exploded view of a representative implantable active electrical system 100 is shown. In the system shown in FIG. 10, implantable active electrical device 10 comprises a connector header 40 configured to receive connector 50 at proximal end of lead extension 30. Of course, it will be understood that device 10 need not have a separate header 40 to receive extension 30. The distal end of extension 30 includes a connector 60 configured to receive proximal end of lead 20. Connector 60 has internal electrical contacts 70 configured to electrically couple extension 30 to lead 20 via electrical contacts 80 disposed on the proximal end portion of lead 20. Electrodes 90 are disposed on distal end portion of lead 20 and are electrically coupled to electrical contacts 80, typically through conductors (not shown). Lead 20 may include any number of electrodes 90, e.g. one, two, three, four, five, six, seven, eight, sixteen, thirty-two, or sixty-four. Typically, each electrode 90 is electrically coupled to a discrete electrical contact 80. While not shown, it will be understood that lead 20 may be directly coupled to active implantable medical device 10 without use of extension 30 or adaptor in some systems 100.

Any suitable active implantable medical device employing leads for transmission or receipt of electrical signals may be employed in accordance with the teachings presented herein. For example, a lead may be associated with an active implantable medical device, such as a hearing implant; a cochlear implant; a sensing or monitoring device; a signal generator such as a cardiac pacemaker or defibrillator, a neurostimulator (such as a spinal. cord stimulator, a brain or deep brain stimulator, a peripheral nerve stimulator, a vagal nerve stimulator, an occipital nerve stimulator, a subcutaneous stimulator, etc.), a gastric stimulator; or the like.

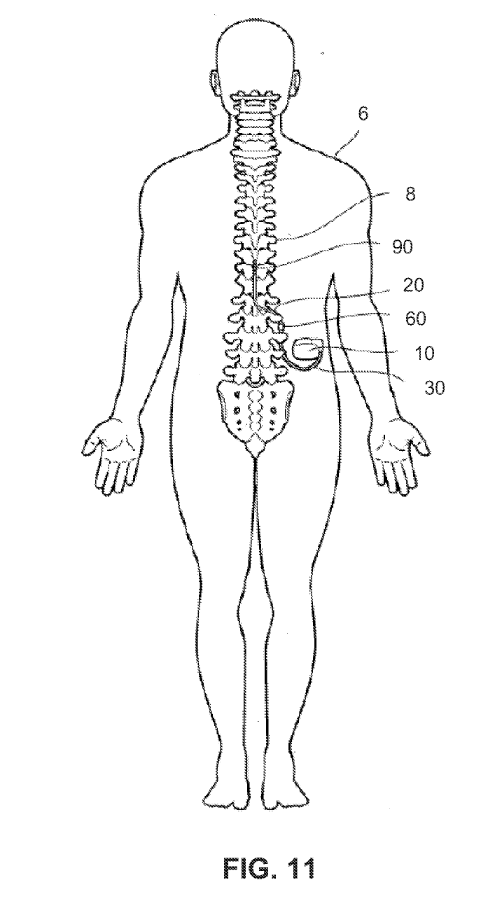

By way of example and referring to FIG. 11, a spinal cord stimulation (SCS) system is shown implanted in a patient 6. For SCS, an implantable pulse generator (IPG) 10 is typically placed in the abdominal region of patient 6 and lead 20 is placed at a desired location along spinal cord 8. Such a system, or any system including an IPG 10 as described herein, may also include a programmer (not shown), such as a physician programmer or a patient programmer. IPG 10 is capable of generating electrical signals that may be applied to tissue of patient 6 via electrodes 90 for therapeutic or diagnostic purposes. IPG 10 contains a power source and electronics for sending electrical signals to the spinal cord 8 via electrodes 90 to provide a desired therapeutic effect.

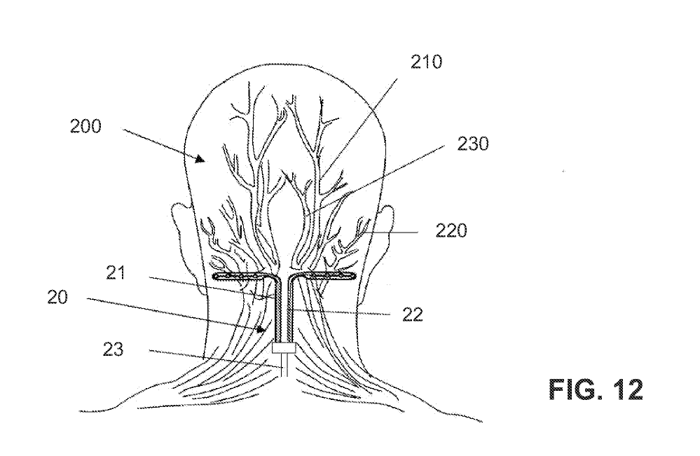

By way of further example and referring to FIG. 12, lead 20 is shown implanted in a patient to provide bilateral therapy to left and right occipital nerves 200. Lead 20 is bifurcated and includes first 21 and second 22 branches forming from a proximal stem portion 23. Of course, two separate leads or lead extensions may be employed for providing electrical signals to occipital nerves 200. As used herein, occipital nerve 200 includes the greater occipital nerve 210, the lesser occipital nerve 220 and the third occipital nerve 230. The greater and lesser occipital nerves are spinal nerves arising between the second and third cervical vertebrae (not shown). The third occipital nerve arises between the third and fourth cervical vertebrae. The portion of the occipital nerve 200 to which an electrical signal is to be applied may vary depending on the disease to be treated and associated symptoms or the stimulation parameters to be applied. In various embodiments, the lead distal portions that contain electrodes are placed to allow bilateral application of electrical signals to the occipital nerve 200 at a level of about C1 to about C2 or at a level in proximity to the base of the skull. The position of the electrode(s) may vary. In various embodiments, one or more electrodes are placed between about 1 cm and about 8 cm from the midline to effectively provide an electrical signal to the occipital nerve 200.

Application of electrical signals to an occipital nerve for treatment of headache, such as migraine, is one particular example or where it may be desirable to employ a lead having a tine distal the electrodes.

Those skilled in the art will recognize that the preferred embodiments may be altered or amended without departing from the true spirit and scope of the disclosure, as defined in the accompanying claims.

What is claimed is:

1. An implantable medical lead comprising:
an proximal body having an axis; and
a distal portion extending from the proximal portion, the distal portion having a changeable width and including
(i) a central body portion including a plurality of electrodes, the plurality of electrodes being aligned with the axis of the proximal body,
(ii) first and second edge portions on opposing sides of the central body portion, the edge portions defining the width of the distal portion and having a collapsed configuration and an expanded configuration,
wherein, when the first and second edge portions are in the collapsed configuration, the distal portion of the lead has a first width and is configured to be received by a lumen of an introducer,
wherein, when the first and second edge portions are in the expanded configuration, the distal portion of the lead has a second width greater than the first width, wherein the second width is equal to or greater than an outer diametric dimension of the introducer,
wherein the first and second edge portions are free of electrodes, and
wherein the first and second edge portions are biased towards the expanded configuration.

2. A lead according to claim 1, wherein the distal portion of the lead further comprises first and second resilient members,
wherein the first resilient member spans a region between the central body and the first edge portion, wherein the first resilient member biases first edge portion towards the expanded configuration, and
wherein the second resilient member spans a region between the central body and the second edge portion, wherein the second resilient member biases the second edge portion towards the expanded configuration.

3. A lead according to claim 1, wherein the first and second edge portions comprise one or more biasing elements that bias the edge portions in the expanded configuration.

4. A lead according to claim 1, further comprising a first flexible membrane spanning the area between the central body portion and the first edge, and a second flexible membrane spanning the area between the central body portion and the second edge.

5. A lead according to claim 1, wherein the first and second edge portions, in the expanded configuration, have ramped distal portions configured to facilitate conversion to the collapsed configuration as the distal portion is pushed into the lumen of the introducer.

6. A lead according to claim 1, wherein the first and second edge portions, in the expanded configuration, have ramped proximal portions configured to facilitate conversion to the collapsed configuration as the distal portion is pulled into the lumen of the introducer.

7. A lead according to claim 1, further comprising a plurality of protrusions disposed along the first and second edge portions.

8. A lead according to claim 7, wherein the protrusions are resilient and have a collapsed configuration and an expanded configuration, and wherein the protrusions are biased in the expanded configuration.

9. A lead according to claim 8, wherein the protrusions have ramped distal portions configured to facilitate conversion to the collapsed configuration as the distal portion is pushed into the lumen of the introducer, and have ramped proximal portions configured to facilitate conversion to the collapsed configuration as the distal portion is pulled into the lumen of the introducer.

10. A system comprising:
a lead introducer comprising a body member defining a lumen, the body member having an inner diametric dimension defined by the lumen and an outer diametric dimension; and
an implantable medical lead having a proximal body having an axis and a distal portion extending from the proximal portion, the distal portion having a changeable width and including
(i) a central body portion including a plurality of electrodes, the plurality of electrodes being aligned with the axis of the proximal body,
(ii) first and second edge portions on opposing sides of the central body portion, the edge portions defining the width of the distal portion and having a collapsed configuration and an expanded configuration,
wherein, when the first and second edge portions are in the collapsed configuration, the distal portion of the lead has a first width and is configured to be received by the lumen of the introducer body member,
wherein, when the first and second edge portions are in the expanded configuration, the distal portion of the lead has a second width greater than the first width, wherein the second width is equal to or greater than an outer diametric dimension of the introducer body member,
wherein the first and second edge portions are free of electrodes, and
wherein the first and second edge portions are biased towards the expanded configuration.

11. A system according to claim 10, wherein the distal portion of the lead further comprises first and second resilient members,
wherein the first resilient member spans a region between the central body and the first edge portion, wherein the first resilient member biases first edge portion towards the expanded configuration, and
wherein the second resilient member spans a region between the central body and the second edge portion, wherein the second resilient member biases the second edge portion towards the expanded configuration.

12. A system according to claim 10, wherein the first and second edge portions comprise one or more biasing elements that bias the edge portions in the expanded configuration.

13. A system according to claim 10, wherein the first and second edge portions of the lead, in the expanded configuration, have ramped distal portions configured to facilitate conversion to the collapsed configuration as the distal portion is pushed into the lumen of the introducer.

14. A system according to claim 10, wherein the first and second edge portions of the lead, in the expanded configuration, have ramped proximal portions configured to facilitate conversion to the collapsed configuration as the distal portion is pulled into the lumen of the introducer.

15. A system according to claim 10, wherein the lead further comprises a plurality of protrusions disposed along the first and second edge portions.

16. An implantable medical lead, comprising:
an elongate body member having a proximal end and a distal end;
a plurality of contacts in proximity to the proximal end;
a plurality of electrodes in proximity to the distal end, each of the plurality of electrodes being electrical coupled to a discrete contact of the plurality of contacts; and
a plurality of protrusions extending from the body in proximity to the distal end, wherein the protrusions are resilient and have a collapsed configuration and an expanded configuration, wherein the protrusions are biased in the expanded configuration,
wherein the elongate body member has a diametric dimension less than the inner diametric dimension defined by a lumen of an introducer such that the body member is capable of being slidably received by the lumen of the introducer,
wherein the lead, in the distal region having the plurality of protrusions, has (i) a diametric dimension equal to or greater than the outer diametric dimension of the introducer when the protrusions are in the expanded configuration, and (ii) a diametric dimension less than or equal to the inner diametric dimension of the introducer when the protrusions are in the collapsed configuration,
wherein the protrusions have ramped distal portions configured to facilitate conversion to the collapsed configuration as the distal portion is pushed into the lumen of the introducer, and have ramped proximal portions configured to facilitate conversion to the collapsed configuration as the distal portion is pulled into the lumen of the introducer.

17. A lead according to claim 16, wherein the elongate lead body is cylindrical and has a generally uniform outer diameter from the proximal end to the distal end.

18. A system comprising:
a lead introducer comprising a body member defining a lumen, the body member having an inner diametric dimension defined by the lumen and an outer diametric dimension; and
an implantable medical lead having
an elongated lead body having a proximal end and a distal end,
a plurality of contacts in proximity to the proximal end;
a plurality of electrodes in proximity to the distal end, each of the plurality of electrodes being electrical coupled to a discrete contact of the plurality of contacts; and
a plurality of protrusions extending from the body in proximity to the distal end,
wherein the protrusions are resilient and have a collapsed configuration and an expanded configuration, wherein the protrusions are biased in the expanded configuration,
wherein the elongate body member has a diametric dimension smaller than the inner diametric dimension of the introducer such that the body member is capable of being slidably received by the lumen of the introducer,
wherein the lead, in the distal region having the plurality of protrusions, has (i) a diametric dimension greater than or equal to the outer diametric dimension of the introducer when the protrusions are in the expanded configuration, and (ii) a diametric dimension less than the inner diametric dimension of the introducer when the protrusions are in the collapsed configuration,
wherein the protrusions have ramped distal portions configured to facilitate conversion to the collapsed configuration as the distal portion is pushed into the lumen of the introducer, and have ramped proximal portions configured to facilitate conversion to the collapsed configuration as the distal portion is pulled into the lumen of the introducer.
